(19) United States
(12) Patent Application Publication
(10) Pub. No.: US 2003/0129150 A1
(43) Pub. Date: Jul. 10, 2003

(54) Cosmetic preparations containing plant extracts

(76) Inventors: Gilles Pauly, Nancy (FR); Philippe Moser, Essey-Les-Nancy (FR); Olga Freis, Seichamps (FR)

Correspondence Address: COGNIS CORPORATION, 2500 RENAISSANCE BLVD., SUITE 200, GULPH MILLS, PA, 19406

(21) Appl. No.: 10/204,941

(22) PCT Filed: Feb. 16, 2001
(86) PCT No.: PCT/EP01/01725

(30) Foreign Application Priority Data
Feb. 25, 2000 (FR) 00/02425

(51) Int. Cl. A61K 7/42; A61K 7/06; A61K 35/78

(52) U.S. Cl. 424/59; 424/74; 424/725; 536/5; 536/18.1

(57) ABSTRACT

The invention relates to extracts of the plant Mourera fluviatilis and to cosmetic and/or pharmaceutical preparations that contain an extract of the plant Mourera fluviatilis.

### FIELD OF THE INVENTION

This invention relates generally to cosmetics and more particularly to preparations containing special plant extracts and to the use of these plant extracts in cosmetic and/or pharmaceutical preparations, for example for treating the skin.

### PRIOR ART

For many years, plant extracts have been used for medicinal and also for cosmetic purposes in various cultures. New plants are always being extracted and the extracts studied for their cosmetic effects in the search to find further plants with a new or different action spectrum. Many plants whose value was not yet known and which were regarded as exotic and unimportant are now widely used inter alia in the cosmetics field.

Today, cosmetic preparations are available to the consumer in a variety of combinations. Consumers not only expect these cosmetics to have a certain care effect or to eliminate a certain deficiency, they are also increasingly demanding products which combine several properties and thus show an improved performance spectrum. There is a particular interest in substances which both positively influence the technical properties of the cosmetic product, such as storage stability, light stability and formulatability, and at the same time represent active principles that impart, for example, caring, moisturizing, irritation-inhibiting, inflammation-inhibiting and/or sun protection properties to the skin and/or hair. In addition, consumers demand high dermatological compatibility and, above all, the use of natural products.

There is also a general demand for cosmetic and pharmaceutical preparations which, by virtue of their special composition, have high-quality technical properties and which, in addition, are distinguished by additional properties for the skin and hair.

### DESCRIPTION OF THE INVENTION

The problem addressed by the present invention was to provide cosmetic and/or pharmaceutical preparations that would meet the requirements for cosmetic formulations, such as storage stability and dermatological compatibility, and in addition would have improved moisture-regulating, caring and protecting properties for human skin and/or hair. Another problem addressed by the invention was to obtain plant extracts from plants which had not hitherto been known for cosmetic applications and to make their ingredients suitable for use as active principles in cosmetic and/or pharmaceutical preparations.

The present invention relates to extracts of the plant Mourera fluviatilis.

Plants in the context of the present invention are understood to be both whole plants and parts thereof (leaves, blossoms, roots) and mixtures thereof.

Mourera fluviatilis

The extracts to be used in accordance with the invention are obtained from plants of the family Podostemaceae and are extracts of the plant Mourera fluviatilis. This plant is one of the “stalk fiber” plants which occur above all in tropical fast-flowing waters and waterfalls. The plant is native to Northern South America and French Guyana where it is also known as “Coumarou salad”. The plant has racemose inflorescences with double-coated bracts. These inflorescences contain 14 to 40 anthers.

Extraction

The extracts to be used in accordance with the invention may be prepared by known methods of extracting plants or parts thereof. Particulars of suitable conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to the expert and which may all be used in principle, can be found for example in Hagers Handbuch der pharmazeutischen Praxis (5th Edition, Vol. 2, pp. 1026-1030, Springer Verlag, Berlin-Heidelberg-New York 1991). Fresh plants or parts thereof are suitable as the starting material although dried plants and/or plant parts which may be mechanically size-reduced before extraction are normally used. Any size reduction methods known to the expert, for example crushing in a mortar, may be used.

Preferred solvents for the extraction process are organic solvents, water (distilled or non-distilled, preferably hot water with a temperature above 80° C.) or mixtures of organic solvents and water, more particularly low molecular weight alcohols, esters, hydrocarbons, ketones or halogenated hydrocarbons with more or less large water contents. Extraction with water, methanol, ethanol, pentane, hexane, heptane, acetone, propylene glycols, polyethylene glycols, ethyl acetate, dichloromethane, trichloromethane and mixtures thereof is particularly preferred. The extraction process is generally carried out at 20 to 100° C., preferably at 30 to 90° C. and more particularly at 60 to 80° C. In one possible embodiment, the extraction process is carried out in an inert gas atmosphere to avoid oxidation of the ingredients of the extract. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of dried plants are in the range from 3 to 20 and more particularly 4 to 16% by weight. The present invention includes the observation that the extraction conditions and the yields of the final extracts may be selected according to the desired application. If desired, the extracts may then be subjected, for example, to spray drying or freeze drying.

The present invention also relates to cosmetic and/or pharmaceutical preparations which contain an extract of the plant Mourera fluviatilis.

Cosmetic and/or pharmaceutical preparations based on the plant Mourera fluviatilis show surprisingly good skin and hare care and protecting properties against stress and again environmental influences coupled with high dermatological compatibility. In addition, the preparations thus obtained are effective moisture-regulating skin moisturizers. The preparations thus obtained are also distinguished by a high antioxidation capacity which, on the one hand, protects the skin against inflammatory reactions and against oxidation-induced skin ageing processes; on the other hand, cosmetic preparations are simultaneously protected against oxidative degradation (deterioration). In addition, the products thus obtained are capable of preventing damage to human fibroblasts and keratinocytes by UV radiation and may therefore be used as sun protection factors in cosmetics.

The quantity of plant extracts used in the preparations mentioned is governed by the concentration of the individual ingredients and by the way in which the extracts are used. In general, the plant extract is used in a quantity—based on the final cosmetic and/or pharmaceutical preparation—of 0.01 to 25% by weight, preferably 0.03 to 10% by weight and more particularly 0.1 to 5% by weight, with the proviso that the quantities add up to 100% by weight with other auxiliaries and additives and with water. The total content of auxiliaries and additives may be 1 to 50% by weight and is preferably 5 to 40% by weight, based on the final cosmetic and/or pharmaceutical preparation. The preparations may be produced by standard cold or hot processes but are preferably produced by the phase inversion temperature method.

Extracts

The extracts of the plant Mourera fluviatilis according to the invention generally contain ingredients from the group consisting of saponins, flavone derivatives, sterols, triterpenes, xanthone derivatives and/or carotinoids. These ingredients differ in composition according to the starting material and extraction method selected.

Saponins in the context of the invention are saponins which can be isolated from the plant Mourera fluviatilis. More particularly, they are a group of glycosides which form colloidal soap-like solutions in water. The saponins are divided into steroid saponins and triterpene saponins according to the nature of their aglycones, the sapogenins.

Flavone derivatives in the context of the invention are understood to be those which can be isolated from the plant Mourera fluviatilis. More particularly, they are hydrogenation, oxidation or substitution products of 2-phenyl-4H-1-benzopyran; hydrogenation may already be present in the 2,3-position of the carbon chain, oxidation may already be present in the 4-position and substitution products are understood to be the replacement of one or more hydrogen atoms by hydroxy or methoxy groups. Accordingly, this definition also encompasses flavans, flavan-3-ols (catechols), flavan-3,4-diols (leucoanthocyanidines), flavones, flavonols and flavonones in the traditional sense.

Sterols in the context of the invention are steroids which can be isolated from the plant Mourera fluviatilis. More particularly, they are steroids which only bear a hydroxy group at C-3, but no other functional group, i.e. formally are alcohols. In addition, the sterols containing 27 to 30 carbon atoms generally have a C═C double bond in the 5/6 position and occasionally even/or in the 7/8, 8/9 and other positions (for example 22/23).

Triterpenes in the context of the invention are triterpenes which can be isolated from the plant Mourera fluviatilis. Formally, the triterpenes according to the invention may be regarded as polymerization products of the hydrocarbon isoprene. The triterpenes (C30) are formed from three isoprene residues. Various polycyclic ring systems for the possible triterpenes may be derived from various folding possibilities of the three isoprene residues. Cyclization mainly provides 6-rings and—with most tetracyclic triterpenes (for example cucurbitacins) and some pentacyclic triterpenes (for example lupans)—also 5-rings. Since the 6-rings are present in chair and boat conformation and since the 5-rings can be flat or angular, many different skeletons are possible.

Xanthone derivatives in the context of the invention are those which can be isolated from the plant Mourera fluviatilis. They are derivatives of the dibenzo-gamma-pyrones. The xanthone derivatives are also referred to as 9-xanthenone derivatives. The xanthone derivatives according to the invention are preferably 6-deoxyjacareubin and/or trapezfoliaxanthone. In addition, the 9-xanthenones may preferably be present as hydroxy- and/or methoxy-substituted xanthenones, such as gentianic acid for example. The derivatives of the xanthones are mostly pale yellow in color and have a light blue fluorescence.

Carotinoids in the context of the invention are those which can be isolated from the plant Mourera fluviatilis. More particularly, they are substances which, chemically, represent 11× to 12× unsaturated tetraterpenes with a basic skeleton containing 9 conjugated double bonds, 8 methyl branches (including the possible ring structures) and a β-ionone structure at one end of the molecule, but which differ in structure at the other end of the molecule. Typical carotinoids are, for example, β-carotene or provitamin A, α-carotene, lutein, cryptoxanthine, zeaxanthine and lycopene. Heinrich et al. report on the use of carotinoids in systemic sun protection in Parf. Kosm. 78,10 (1997).

In one particular embodiment of the invention, the extracts of the plant Mourera fluviatilis contain minerals in the form of salts of the alkali or alkaline earth metals. The metals predominantly occurring are sodium, potassium or calcium. The alkali or alkaline earth metals occur in the form of their salts, but mainly in the form of their halides, oxides or hydroxides, phosphates, carbonates, sulfates or nitrates.

The present invention includes the observation that particularly effective cosmetic preparations are obtained through the co-operation of the ingredients of the plant extracts, especially those mentioned above.

The present invention also relates to the manifold use of the plant extracts of Mourera fluviatilis, for example

in skin and hair care preparations, particularly against stress;

in moisture-regulating moisturizers.

Care Preparations

Care preparations in the context of the invention are understood to be hair and skin care preparations. These care preparations have inter alia a cleaning and restoring effect and show moisture-regulating and UV protection properties. In principle, the extracts according to the invention may be used in any cosmetic products. Examples of cosmetic products are described in the form of their formulations in Table 7 to Table 15.

The object of hair care is to keep freshly regrown hair in its natural state for as long as possible or to repair it in the event of damage. Features of naturally healthy hair are a silky sheen, low porosity, springy and soft body and a pleasantly smooth feel (good “feel”). The care preparations according to the invention have a smoothing effect on the hair and improve its combability, prevent electrostatic charging and improve feel and sheen.

The preparations according to the invention have an excellent skin-care effect coupled with high dermatological compatibility. They also show high stability, particularly against oxidative decomposition of the products.

Moisturizers

Moisture-regulating moisturizers according to the invention are understood to be skin care preparations which are intended to regulate skin moisture. In the context of the invention, this conforms to the definition of a moisturizer. They are substances or mixtures of substances which provide cosmetic and/or pharmaceutical preparations with the ability to reduce the release of moisture from the Stratum corneum (horny layer) after application to and spreading over the surface of the skin.

The moisturizers according to the invention contain extracts of the plant Mourera fluviatilis. Other moisturizers, for example, may be present in combination with the plant extract, including:

polyglycerol fatty acid esters based on C₁₂₋₁₈ fatty acids, for example tetraglyceryl monooleate, triglyceryl diisostearate;

pyroglutamic acid or L-arginine pyroglutamate, L-lysine pyroglutamate;

mixtures of amino acids such as, for example, L-alanine, L-arginine, L-serine, L-threonine;

propylene glycol

acetamide

polysaccharides or hyaluronic acid;

castor oil ethers and sorbitan esters as described in JP 60149511 (Lion Corp.).

Sun (UV) Protection Factors

The present invention also relates to the use of the extracts of the plant Mourera fluviatilis in sun protection preparations.

Sun protection factors or UV protection factors in the context of the invention are light protection factors which are useful in protecting human skin against harmful effects of direct and indirect solar radiation. The ultraviolet radiation of the sun responsible for tanning of the skin is divided into the sections UV-C (wavelengths 200-280 nm), UV-B (280-315 nm) and UV-A (315-400 nm).

The pigmenting of normal skin under the influence of solar radiation, i.e. the formation of melanins, is differently effected by UV-B and UV-A. Exposure to UV-A (long-wave UV) results in darkening of the melanins already present in the epidermis without any sign of harmful effects. It is different with so-called short-wave UV (UV-B). This promotes the formation of so-called late pigment through the reformation of melanins. However, before the (protective) pigment is formed, the skin is exposed to the unfiltered radiation which, depending on the exposure time, can lead to reddening of the skin (erythema), inflammation of the skin (sunburn) or even blisters.

Extracts of the plant Mourera fluviatilis are used as UV absorbers or light filters which convert UV radiation into harmless heat. They may additionally be present in combination with other sun protection factors or UV protection factors.

These other UV protection factors are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:

3-benzylidene camphor or 3-benzylidene norcamphor and derivatives thereof, for example 3-(4-methylbenzylidene)-camphor as described in EP-B1 0693471;

4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;

esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

esters of salicylic acid, preferably salicylic acid-2-ethylhexyl ester, salicylic acid-4-isopropylbenzyl ester, salicylic acid homomenthyl ester;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably 4-methoxybenzmalonic acid di-2-ethylhexyl ester;

triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (Uvasorb® HEB);

propane-1,3-diones such as, for example, 1-(4-tert.butylphenyl)-3-(4′-methoxyphenyl)-propane-1,3-dione;

ketotricyclo(5.2.1.0)decane derivatives as described in EP 0694521 B1.

Suitable water-soluble substances are

2-phenylbenzimidazole-5-sulfonic acid and alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and salts thereof;

sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 197 12 033 A1 (BASF). The UV-A and UV-B filters may of course also be used in the form of mixtures. Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or dimethicones. So-called micro- or nanopigments are preferably used in sun protection products. Micronized zinc oxide is preferably used. Other suitable UV filters can be found in P. Finkel's review in SÖFW-Journal 122, 543 (1996) and in Parfümerie und Kosmetik 3 (1999), pages 11 et seq.

The present invention also relates to the use of extracts of the plant Mourera fluviatilis in preparations against fibroblast and/or keratinocyte damage by UV-A radiation and/or UV-B radiation and as anti-inflammatory additives.

UV-A rays penetrate into the dermis where they lead to oxidative stress which is demonstrated by lipoperoxidation of the cytoplasm membranes. The lipoperoxides are degraded to malonaldialdehyde (MDA) which will crosslink many biological molecules, such as proteins and nuclein bases (enzyme inhibition or mutagenesis). The extracts of the plant Mourera fluviatilis according to the invention significantly reduce the level of MDA in human fibroblasts induced by UV-A rays and thus show a high capacity for reducing the harmful effects of oxidative stress on the skin.

UV-B rays initiate inflammation by activating an enzyme, namely phospholipase A2 or PLA2. This inflammation (erythema, odema) is indued by the removal of arachidonic acid from the phospholipids of the plasma membrane by the phospholipase. Arachidonic acid is the precursor of the prostaglandins which cause inflammation and cell membrane damage. The prostaglandins E2 (=PGE2) are formed by cyclooxygenase. The degree of release of the cytoplasm enzyme LDH (lactate dehydrogenase) in human keratinocytes serves as a marker for cell damage.

The extracts of the plant Mourera fluviatilis reduce the effect of UV-B radiation on the number of keratinocytes and on the content of released LDH. Accordingly, the extracts have the ability to reduce cell membrane damage caused by UV-B radiation.

In principle, the extracts according to the invention may be used as anti-inflammatory additives for any cosmetic and/or pharmaceutical preparations used against inflammation of the skin and hence in skin care. The inflammation of the skin may be caused by various factors.

The present invention also relates to the use of extracts of the plant Mourera fluviatilis as antioxidants or radical traps.

Antioxidants in the context of the invention are oxidation inhibitors which can be isolated from the plant Mourera fluviatilis. Antioxidants are capable of inhibiting or preventing changes caused by the effects of oxygen and other oxidative processes in the substances to be protected. The effect of antioxidants consists mainly in their acting as radical traps for the free radicals occurring during autoxidation.

Besides the use of extracts of the plant Mourera fluviatilis as antioxidants, other already known antioxidants may also be used. One possible use of the antioxidants, for example in cosmetic and/or pharmaceutical preparations, is their use as secondary sun protection factors because antioxidants are capable of interrupting the photochemical reaction chain which is initiated when UV rays penetrate into the skin. Besides the plant extract according to the invention, typical examples are amino acids (for example glycine, alanine, arginine, serine, threonine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene, lutein) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, boldin, boldo extract, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

The UV protection factors or antioxidants may be added in quantities of 0.01 to 25, preferably 0.03 to 10 and more particularly 0.1 to 5% by weight, based on the total quantity in the preparations.

The extracts according to the invention may be used in cosmetic and/or pharmaceutical preparations such as, for example, hair shampoos, hair lotions, foam baths, shower baths, creams, gels, lotions, alcoholic and aqueous/alcoholic solutions, emulsions, wax/fat compounds, stick preparations, powders or ointments. These preparations may also contain mild surfactants, oil components, emulsifiers, pearlizing waxes, consistency factors, thickeners, superfatting agents, stabilizers, polymers, silicone compounds, fats, waxes, lecithins, phospholipids, biogenic agents, UV protection factors, antioxidants, deodorants, antiperspirants, antidandruff agents, film formers, swelling agents, insect repellents, self-tanning agents, tyrosine inhibitors (depigmenting agents), hydrotropes, solubilizers, perservatives, perfume oils, dyes and the like as further auxiliaries and additives.

Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Suitable oil components are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols, esters of branched C₆₋₁₃ carboxylic acids with linear C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols (cf. DE 197 56 377 A1), more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di-and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, Guerbet carbonates, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Suitable emulsifiers are, for example, nonionic surfactants from at least one of the following groups:

products of the addition of 2 to 30 moles of ethylene oxide and/or 0 to 5 moles of propylene oxide onto linear C₈₋₂₂ fatty alcohols, C₁₂₋₂₂ fatty acids and alkyl phenols containing 8 to 15 carbon atoms in the alkyl group and alkylamines containing 8 to 22 carbon atoms in the alkyl group;

alkyl and/or alkenyl oligoglycosides containing 8 to 22 carbon atoms in the alkyl group and ethoxylated analogs thereof;

adducts of 1 to 15 moles of ethylene oxide with castor oil and/or hydrogenated castor oil;

adducts of 15 to 60 moles of ethylene oxide with castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 moles of ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 moles of ethylene oxide;

mixed esters of pentaerythritol, fatty acids, citric acid and fatty alcohol according to DE 11 65 574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethylene glycol-30 Dipolyhydroxystearate;

polymer emulsifiers, for example Pemulen types (TR-1, TR-2) of Goodrich;

polyalkylene glycols and

glycerol carbonate.

The addition products of ethylene oxide and/or propylene oxide with fatty alcohols, fatty acids, alkylphenols or with castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as refatting agents for cosmetic formulations from DE 20 24 051 PS.

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 moles of ethylene oxide with the partial glycerides mentioned are also suitable.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 moles of ethylene oxide with the sorbitan esters mentioned are also suitable.

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 moles of ethylene oxide.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethylimidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine.

Finally, cationic surfactants are also suitable emulsifiers, those of the esterquat type, preferably methyl-quaternized difatty acid triethanolamine ester salts, being particularly preferred.

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs) and correspond to the following general formula:

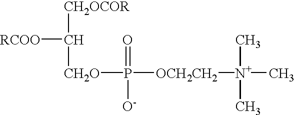

where R typically represents linear aliphatic hydrocarbon radicals containing 15 to 17 carbon atoms and up to 4 cis-double bonds. Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycero-phosphates) which are normally classed as fats. Sphingosines and sphingolipids are also suitable.

Suitable pearlescing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxy-substituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Metal salts of fatty acids such as, for example, magnesium, aluminium and/or zinc stearate or ricinoleate may be used as stabilizers.

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinylimidazole polymers such as, for example, Luviquat® (BASF), condensation products of polyglycols and amines, quaternized collagen polypeptides such as, for example, Lauryidimonium Hydroxypropyl Hydrolyzed Collagen (Lamequat® L, Grünau), quaternized wheat polypeptides, polyethyleneimine, cationic silicone polymers such as, for example, Amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2 252 840 A and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol®) AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosmetics & Toiletries, Vol. 108, May 1993, pages 95 et seq.

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

In the context of the invention, biogenic agents are, for example, tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, other plant extracts and vitamin complexes.

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monolaurate (GML), diglycerol monocaprate (DMC), salicylic acid-N-alkylamides such as, for example, salicylic acid-n-octyl amide or salicylic acid-n-decyl amide.

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT, Henkel KGaA, Düsseldorf, FRG). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexyl-cinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romilat, irotyl and floramat.

Antiperspirants reduce perspiration and thus counteract underarm wetness and body odor by influencing the activity of the eccrine sweat glands. Aqueous or water-free antiperspirant formulations typically contain the following ingredients:

astringent active principles,

oil components,

nonionic emulsifiers,

co-emulsifiers,

consistency factors,

auxiliaries in the form of, for example, thickeners or complexing agents and/or

nonaqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,

inflammation-inhibiting, skin-protecting or pleasant-smelling essential oils,

synthetic skin-protecting agents and/or

oil-soluble perfume oils.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Suitable antidandruff agents are Pirocton Olamin (1-hydroxy-4-methyl-6-(2,4,4-trimethylpentyl)-2-(1H )-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxy-phenyl}-piperazine, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Suitable insect repellents are N,N-diethyl-m-toluamide, pentane-1,2-diol or Ethyl Butylacetylaminopropionate.

A suitable self-tanning agent is dihydroxyacetone. Suitable tyrosine inhibitors which prevent the formation of melanin and are used in depigmenting agents are, for example, arbutin, koji acid, coumaric acid and ascorbic acid (vitamin C).

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Suitable preservatives are, for example, phenoxyethanol, formaldehyde solution, parabens, pentanediol or sorbic acid and the other classes of compounds listed in Appendix 6, Parts A and B of the Kosmetikverordnung (ACosmetics Directive≅).

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamon, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable dyes are any of the substances suitable and approved for cosmetic purposes as listed, for example, in the publication AKosmetische Färbemittel≅of the Farbstoffkommission der Deutschen Forschungs-gemeinschaft, Verlag Chemie, Weinheim, 1984, pages 81 to 106. These dyes are normally used in concentrations of 0.001 to 0.1% by weight, based on the mixture as a whole.

The present invention also relates to a process for preparing an extract of the plant Mourera fluviatilis in which solvents or mixtures of solvents selected from the group consisting of distilled or nondistilled water, low molecular weight alcohols, esters or hydrocarbons are used for extraction of the plant.

### EXAMPLES

### Example 1

300 g dried Mourera fluviatilis plants were coarsely crushed in a mortar and then transferred to a glass reactor. 3 liters distilled water were then poured on. The infusion was heated to between 80 and 85° C. and extracted at that temperature with stirring for 1 hour. The mixture was then cooled to 20° C. and centrifuged for 15 mins. at a speed of 5000 G. The supernatant colloidal liquid was separated from the residue by filtration through depth filters with a mean porosity of 0.450 μm (from Seitz of Bordeaux, France), 1.7 liters extract with a dry residue of 2.4% by weight being obtained. The brown-colored extract was spray-dried at a starting temperature of 185° C. and an end temperature of 80° C. The yield of dry product was 13.5% by weight, based on the dry weight of plants used.

### Example 2

The procedure was as in Example 1 except that, after centrifuging, the aqueous extract was adjusted to pH 7.2±0.2 and filtered by filtration through depth filters with a mean porosity of 0.200 μm.

### Example 3

Example 1 was repeated except that extraction was carried out with 3 liters 80% by weight aqueous methanol. Extraction was carried out with stirring under reflux for 1 hour at boiling temperature and the extract was further processed as described above. Filtration was carried out as described in Example 1. Thereafter the alcohol was removed under reduced pressure at 45° C. and the green-brown residue was then spray-dried as described. The yield of dry product was 15.9% by weight, based on the dry weight of plants used.

### Example 4

Example 1 was repeated except that extraction was carried out with 3 liters of 96% by weight aqueous ethanol. Extraction was carried out with stirring under reflux for 1 hour at boiling temperature and the extract was further processed as described above. Filtration was carried out as described in Example 1. Thereafter the alcohol was removed under reduced pressure at 45° C. and the green residue was then spray-dried at 50° C. The yield of dry product was 4.9% by weight, based on the dry weight of plants used.

### Example 5

260 g of the dried residue from Example 4 were transferred to a glass reactor into which 2.6 liters distilled water were then poured. The infusion was further treated as described in Example 1. The brown-colored extract was spray-dried at a starting temperature of 185° C. and an end temperature of 80° C. The yield of dry product was 12.5% by weight, based on the dry weight of dried residue used.

### Example 6

### Activity Towards Free Radicals

In a first series of tests, the extracts were investigated for their performance against oxidative stress. The extracts of Examples 1 to 5 were used in concentrations of 0% w/v (weight per volume); 0.03% w/v and 0.1% w/v. In a first test, the hydroxylation of salicylic acid by hydroxyl radicals (from the reaction of hydrogen peroxide with iron(III) ions and EDTA) was investigated as a reference system. This reaction can be photometrically investigated because the hydroxylation product of salicylic acid is reddish in color. The influence of the extracts on the formation of hydroxysalicylic acid was measured at an optical density of 490 nm. The results are set out in Table 1 as the degree of hydroxysalicylic acid formation in %-absolute.

TABLE 1
Degree of hydroxylation of salicylic acid
in %-absolute (results are
averages of two measurements)

| Concentration | Extract of | Extract of | Extract of | Extract of |
|---|---|---|---|---|
| (% w/v) | Example 1 | Example 3 | Example 4 | Example 5 |
| 0.0 | 100 | 100 | 100 | 100 |
| 0.03 | 75 | 95 | 76 | 85 |
| 0.1 | 33 | 77 | 36 | 46 |
| IC₅₀ % w/v | 0.072 | 0.188 | 0.076 | 0.093 |

The results set out in Table 1 show that the extracts of the plant Mourera fluviatilis used are active against radicals. Extracts differing in their activity according to the extraction process used are obtained. Using the extraction process described in Example 1 for example, a concentration of 0.072% w/v is sufficient to obtain a 50% inhibition of the radical reaction. In this case, the formation of hydroxysalicylic acid by hydroxy radicals is reduced by 50% at that concentration.

In a third test, xanthine oxidase was selected as the test system. Under oxidative stress, the enzyme converts purine bases, such as adenine or guanine for example, into uronic acid. The oxygen radicals formed as intermediates can be detected and quantitatively determined by reaction with luminol (through the luminescence). The luminescence yield decreases in the presence of substances with radical-trapping properties.

TABLE 2
Luminescence inhibition in %-absolute

| Concentration | Extract of | Extract of | Extract of | Extract of |
|---|---|---|---|---|
| (% w/v) | Example 1 | Example 3 | Example 4 | Example 5 |
| 0.0 | 0 | 0 | 0 | 0 |
| 0.03 | 11 | 33 | 27 | — |
| 0.1 | 71 | 76 | 60 | 36 |
| IC₅₀ % w/v | 0.076 | 0.058 | 0.079 | 0.127 |

It can be seen from Table 2 that the extracts of the plant Mourera fluviatilis inhibit the radical-induced formation of luminescence. Various degrees of inhibition are obtained according to the concentration and the extraction process used. A concentration of only 0.058% w/v of the extract of Example 3 provides 50% inhibition of luminescence and accordingly shows distinct radical-trapping properties.

### Example 7

### Cell Protecting Effect Against UV-A on Human Fibroblasts Cultivated in vitro

Background: UV-A rays penetrate into the dermis where they lead to oxidative stress which is demonstrated by lipoperoxidation of the cytoplasm membranes.

The lipoperoxides are degraded to malonaldialdehyde which will crosslink many biological molecules, such as proteins and nuclein bases (enzyme inhibition or mutagenesis).

Method: To carry out these tests, a defined culture medium containing the fibroblasts is inoculated with foetal calf serum and added to the plant extract (in the defined medium containing 10% foetal calf serum) 72 hours after inoculation.

After incubation for 48 hours at 37° C./5% CO₂, the culture medium was replaced by saline solution (physiological NaCl solution) and the fibroblasts were exposed to a dose of UV-A (365 nm, 15 J/cm²; tubes: MAZDA FLUOR TFWN40).

After the exposure to UV-A, the MDA level (malonaldialdehyde level) in the supernatant sodium chloride solution was quantitatively determined by reaction with thiobarbituric acid.

TABLE 3
Quantification of malonaldialdehyde in
fibroblasts (results in %,
based on the control, average value of
2 tests each repeated 3 times)

| Concentration | Extract of | Extract of | Extract of |
|---|---|---|---|
| (% weight/volume) | Example 3 | Example 4 | Example 5 |
| Control without UV | 0 | 0 | 0 |
| UV-A (365 nm) | 100 | 100 | 100 |
| UV-A + extract 0.01% | — | 65 | — |
| UV-A + extract 0.02% | — | 72 | — |
| UV-A + extract 0.05% | 58 | — | 65 |
| UV-A + extract 0.1% | 57 | — | 74 |

The results set out in Table 3 show that the extracts of the plant Mourera fluviatilis according to the invention significantly reduce the level of MDA in human fibroblasts induced by UV-A rays. These results reflect a high capacity on the part of Mourera fluviatilis extracts to reduce harmful effects of oxidative stress on the skin.

### Example 8

### Effectiveness in Protecting Cells Against UV-B in Human Keratinocytes Cultivated in vitro

Background: UV-B rays cause inflammation (erythema, odema) by activating an enzyme, namely phospholipase A2 or PLA2, which removes arachidonic acid from the phospholipids of the plasma membranes. Arachidonic acid is the precursor of the prostaglandins which cause inflammation and cell membrane damage; the prostaglandins E2 (=PGE2) are formed by cyclooxygenase.

Method: The effect of UV-B radiation was investigated in vitro in keratinocytes by determining the release of the cytoplasm enzyme LDH (lactate dehydrogenase). This enzymes serves as a marker for cell damage.

To carry out the tests, a defined medium containing foetal calf serum was inoculated with the keratinocytes and the plant extract (diluted with saline solution) was added 72 hours after the inoculation.

The keratinocytes were then exposed to a dose of UV-B (30 mJ/cm²-tubes: DUKE GL40E).

After incubation for another day at 37° C./5% CO₂, the LDH content in the supernatant was determined. The LDH (lactate dehydrogenase) content was determined by an enzyme reaction (kit used to determine LDH levels from Roche).

The number of adhering keratinocytes was determined (after trypsin treatment) with a particle counter.

TABLE 4
Cell protecting effect of an extract of
Mourera fluviatilis again UV-B
rays; results in % based on the control,
average value of 2 tests each
repeated 3 times.

| | | Content of LDH |
|---|---|---|
| Extract of Example 3 | Number of keratinocytes | released |
| Control without UV | 100 | 0 |
| UV-B (315 nm) | 49 | 100 |
| UV-B + extract 0.03% | 90 | 44 |
| UV-A + extract 0.1% | 19 | 19 |

The results of these tests show that an extract of the plant Mourera fluviatilis according to the invention reduces the effect of UV-B radiation on the number of keratinocytes and on the content of LDH released. Accordingly, the described extracts have the ability to reduce cell membrane damage caused by UV-B radiation.

### Example 9

### Sensory Activity on Human Hair

The modification of sensory properties of human hair after treatment with extracts of the plant Mourera fluviatilis was evaluated on standardized hair tresses (length 15 cm, weight 3 g). An aqueous sodium lauryl sulfate solution (15% w/v) was used as standard and placebo. The samples of the plant extracts were incorporated in the sodium lauryl sulfate solution and tested in a concentration of 1.5% w/v. The effects of the treatment were investigated in three different types of hair:

a) Control hair: the tresses were washed with an aqueous sodium lauryl sulfate solution (15% w/v).

b) Bleached hair. the tresses were treated for 30 mins. with a bleaching shampoo containing 6% H₂O₂ and ammonia (Éclair clair of L'Oréal) and then washed with the aqueous sodium lauryl sulfate solution (15% w/v). This bleaching process was repeated twice.

c) Permanent-waved (“permed”) hair: the tresses were treated for 20 mins. with a sodium mercaptoacetate solution (6% w/v, pH 6), rinsed and then treated for 10 mins. with an H₂O₂ solution (pH 3). After this solution had been rinsed out, the tresses were washed with aqueous sodium lauryl sulfate solution (15% w/v). This permanent waving cycle was repeated twice.

The hair tresses thus prepared were held in the solution containing the particular test substance for 3 minutes and were then rinsed for 1 minute. After rinsing, the hair tresses were combed and tested for wet combability. The tresses were dried at room temperature. The sensory tests were carried out on the dry hair 24 hours after the treatment with the extracts.

The following properties were determined on the dry hair: combability, flexibility and softness, electrostatic charging, volume and luster.

The results of the sensory tests on wet and dry hair are set out in the following Table. The sensory properties should be read in comparison with standardized hair tresses. The higher the number shown, the better the evaluation of the particular sensory property.

TABLE 5
Sensory properties of human hair
tresses after treatment with
Mourera fluviatilis extracts by
comparison with untreated hair tresses

| Parameter | Control hair | Bleached hair | Permed hair |
|---|---|---|---|
| Combability, wet hair | 2 | 1 | 0 |
| Combability, dry hair | 0 | 0 | 1 |
| Flexibility and softness | 2 | 0 | 1 |
| Electrostatic charging | 2 | 1 | 0 |
| Volume | 1 | 1 | 0 |
| Luster | 2 | 0 | 0 |

The test results show that an extract of the plant Mourera fluviatilis improves the sensory properties of human hair. A significant improvement in the combability of wet hair, softness, volume and luster was established for the control hair after the treatment. Electrostatic charging decreased after treatment with the plant extract.

An improvement in the combability of wet hair and in volume and a reduction in electrostatic charging were established for bleached hair. For permed hair, there was an improvement in dry combability and in softness.

### Example 10

### Skin Moisture Regulation Test

Background: The epidermis of human skin contains the horny layer (the Stratum corneum). The Stratum corneum is a dielectric medium of low electrical conductivity. The water content leads to the increased dielectric conductivity so that determining the dielectric conductivity of the stratum corneum can serve as a measure of the moisture content of human skin. An increase in the dielectric conductivity of the Stratum corneum reflects an increased moisture content of human skin.

Method: The following formulation is presented as an example of cosmetic preparations that were used to the test the moisture-regulating properties of the formulation.

| | |
|---|---|
| pyroglutamic acid: | 11.2% |
| L-alanine: | 7.2% |
| L-arginine: | 17.0% |
| L-serine: | 20.5% |
| L-threonine: | 3.1% |
| dry extract of Example 2: | 1.0% |
| sucrose: | 40.0% |

Samples of normal skin obtained from plastic surgery were used for the test. The Stratum corneum from these skin samples was stored in chambers with defined relative humidity (44%, saturated potassium carbonate solution) and standardized. Each sample of the Stratum corneum was comparatively tested under four conditions:

1) no treatment;

2) placebo treatment;

3) treatment with a preparation consisting of a hydrogel (Hydrogel LS from Laboratoire Sérobiologique LS) containing 2% w/v of the moisture-regulating preparations described above;

4) treatment with a preparation consisting of a hydrogel (Hydrogel LS from Laboratoire Sérobiologique LS) containing 4% w/v of the moisture-regulating preparations described above;

The placebo was the hydrogel (Hydrogel LS Laboratoire Sérobiologique LS) without the described preparation, i.e. without plant extract.

The moisture-regulating activity of the above-described preparation was determined as a percentage increase in conductivity by comparison with the placebo treatment.

The results reflect a dose-dependent moisture-regulating activity.

TABLE 6
Moisture-regulating effect as determined by measurement of
dielectric conductivity (in μS); average
value of 18 measurements (the
standard deviation is shown in brackets)

| | Before | | | | | | |
|---|---|---|---|---|---|---|---|
| Treatment | the treatment | 30 Mins. | 1 Hour | 2 Hours | 4 Hours | 6 Hours | 24 Hours |
| Control | 25.37 | 29.37 | 29.00 | 27.96 | 28.70 | 27.61 | 27.83 |
| | (3.07) | (2.38) | (2.35) | (2.19) | (2.08) | (2.54) | (2.26) |
| Placebo | 28.48 | 46.78 | 37.74 | 30.81 | 31.85 | 31.13 | 30.54 |
| | (2.83) | (2.50) | (2.02) | (1.84) | (1.35) | (1.63) | (1.40) |
| 3) | 26.91 | 62.39 | 49.49 | 41.26 | 36.74 | 33.87 | 32.54 |
| | (2.11) | (4.85) | (2.71) | (2.49) | (2.38) | (2.06) | (1.91) |
| 4) | 28.6 | 72.2 | 56.6 | 44.2 | 40.8 | 36.8 | 37.1 |
| | (2.5) | (3.7) | (2.3) | (3.0) | (3.1) | (1.8) | (2.0) |

The above results show that the Mourera fluviatilis extracts investigated and tested have the following abilities:

they trap and neutralize radicals and reactive forms of oxygen;

they reduce the degree of lipoperoxidation induced by UV-A rays in human fibroblasts;

they reduce the cell damage induced by UV-B in human keratinocytes;

with regard to cosmetics, sensory tests on human hair revealed a distinct restoring, softening and luster-increasing effect and improved combability;

a preparation containing extracts of the plant Mourera fluviatilis showed distinct moisture-regulating properties.

Various cosmetic products or cosmetic preparations containing an extract of the plant Mourera fluviatilis are described in the following as practical embodiments of the invention.

A number of formulation examples are set out in the following Tables.

### Example 11

### Exemplary Formulations of Cosmetic Products Containing Extracts of the Plant Mourera fluviatilis.

The Mourera fluviatilis extracts obtained in accordance with Examples 1 to 5 are used in the following formulations according to the invention K1 to K21 and 1 to 40. Unless otherwise explicitly indicated, any extract of Examples 1 to 5 may be used. The cosmetic preparations thus produced show very good skin-care properties coupled with high dermatological compatibility in relation to the comparison formulations (C1, C2 and C3). In addition, the preparations according to the invention proved to be stable to oxidative decomposition.

TABLE 7
Soft cream formulations K1 to K7
(All quantities in % by weight,
based on the cosmetic preparation)

| INCI name | K1 | K2 | K3 | K4 | K5 | K6 | K7 | C1 |
|---|---|---|---|---|---|---|---|---|
| Glyceryl Stearate (and) | 8.0 | 8.0 | 8.0 | 8.0 | 8.0 | 8.0 | 8.0 | 8.0 |
| Ceteareth-12/20 (and) | | | | | | | | |
| Cetearyl Alcohol (and) Cetyl | | | | | | | | |
| Palmitate | | | | | | | | |
| Cetearyl Alcohol | 2.0 | 2.0 | 2.0 | 2.0 | 2.0 | 2.0 | 2.0 | 2.0 |
| Dicaprylyl Ether | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl Isononanoate | 3.0 | 3.0 | 3.0 | 3.0 | 3.0 | 3.0 | 3.0 | 3.0 |
| Glycerin (86% by weight) | 3.0 | 3.0 | 3.0 | 3.0 | 3.0 | 3.0 | 3.0 | 3.0 |
| Mourera fluviatilis extracts | 0.5 | 0.5 | 0.5 | 0.5 | 0.5 | 0.5 | 0.5 | — |
| (Examples 1-4) | | | | | | | | |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

TABLE 8
Night cream formulations K8 to K14
(All quantities in % by weight,
based on the cosmetic preparation)

| INCI name | K8 | K9 | K10 | K11 | K12 | K13 | K14 | C2 |
|---|---|---|---|---|---|---|---|---|
| Polyglyceryl-2 Dipolyhydroxystearate | 4.0 | 4.0 | 4.0 | 4.0 | 4.0 | 4.0 | 4.0 | 5.0 |
| Polyglyceryl-3 Diisostearate | 2.0 | 2.0 | 2.0 | 2.0 | 2.0 | 2.0 | 2.0 | 2.0 |
| Cera Alba | 2.0 | 2.0 | 2.0 | 2.0 | 2.0 | 2.0 | 2.0 | 2.0 |
| Zinc Stearate | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl Isononanoate | 8.0 | 8.0 | 8.0 | 8.0 | 8.0 | 8.0 | 8.0 | 8.0 |
| Dicaprylyl Ether | 5.0 | 5.0 | 5.0 | 5.0 | 5.0 | 5.0 | 5.0 | 5.0 |
| Magnesium sulfate | 1.0 | 1.0 | 1.0 | 1.0 | 1.0 | 1.0 | 1.0 | 1.0 |
| Glycerin (86% by weight) | 5.0 | 5.0 | 5.0 | 5.0 | 5.0 | 5.0 | 5.0 | 5.0 |
| Mourera fluviatilis extract | 0.5 | 0.5 | 0.5 | 0.5 | 0.5 | 0.5 | 0.5 | — |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

TABLE 9
W/O body lotion formulations K15 to K21.
(All quantities in % by weight,
based on the cosmetic preparation)

| INCI name | K15 | K16 | K17 | K18 | K19 | K20 | K21 | C3 |
|---|---|---|---|---|---|---|---|---|
| PEG-7 Hydrogenated Castor Oil | 7.0 | 7.0 | 7.0 | 7.0 | 7.0 | 7.0 | 7.0 | 7.0 |
| Decyl Oleate | 7.0 | 7.0 | 7.0 | 7.0 | 7.0 | 7.0 | 7.0 | 7.0 |
| Cetearyl Isononanoate | 7.0 | 7.0 | 7.0 | 7.0 | 7.0 | 7.0 | 7.0 | 7.0 |
| Glycerin (86% by weight) | 5.0 | 5.0 | 5.0 | 5.0 | 5.0 | 5.0 | 5.0 | 5.0 |
| MgSO₄.7H₂O | 1.0 | 1.0 | 1.0 | 1.0 | 1.0 | 1.0 | 1.0 | 1.0 |
| Mourera fluviatilis extracts | 1.5 | 1.5 | 1.5 | 1.5 | 1.5 | 1.5 | 1.5 | — |
| (Examples 1-4) | | | | | | | | |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

¹⁾Deoxyribonucleic acid: molecular weight ca. 70,000, purity (determined by spectrophotometric measurement of absorption at 260 nm and 280 nm): at least 1.7

TABLE 10
Formulations for conditioners I
Cosmetic preparations conditioner (water,
preservative to 100% by weight)

| Composition (INCI) | 1 | 2 | 3 | 4 | 5 | 6 |
|---|---|---|---|---|---|---|
| Dehyquart ® A | 4.0 | 4.0 | | | 3.0 | |
| Cetrimonium Chloride | | | | | | |
| Dehyquart L ® 80 | | | 1.2 | 1.2 | | 1.0 |
| Dococoylmethylethoxymonium Methosulfate | | | | | | |
| (and) Propyleneglycol | | | | | | |
| Eumulgin ® B2 | 0.8 | | — | 0.8 | — | 1.0 |
| Ceteareth-20 | | | | | | |
| Eumulgin ® VL 75 | — | 2.0 | 2.0 | — | 0.8 | — |
| Lauryl Glucoside (and) Polyglyceryl-2 | | | | | | |
| Polyhydroxystearate (and) Glycerin | | | | | | |
| Lanette ® O | 3.0 | 3.0 | 3.0 | 3.0 | 3.0 | 3.0 |
| Cetearyl Alcohol | | | | | | |
| Cutina ® GMS | — | 0.5 | — | 0.5 | — | 1.0 |
| Glyceryl Stearate | | | | | | |
| Lamesoft ® PO 65 | | — | 3.0 | — | — | 3.0 |
| Coco-Glucoside (and) Gyceryl Oleate | | | | | | |
| Cetiol ® J 600 | — | 0.5 | — | 1.0 | — | 1.0 |
| Oleyl Erucate | | | | | | |
| Eutanol ® G | — | — | 1.0 | — | — | 1.0 |
| Octyldodecanol | | | | | | |
| Nutrilan ® Keratin W | 5.0 | — | — | 2.0 | — | — |
| Hydrolyzed Keratin | | | | | | |
| Generol ® 122 N | — | — | — | — | 1.0 | 1.0 |
| Soya Sterol | | | | | | |
| Mourera fluviatilis extract | 1.0 | 1.0 | 1.0 | 1.0 | 1.0 | 1.0 |
| Copherol ® 12250 | — | — | 0.1 | 0.1 | — | — |
| Tocopherol Acetate | | | | | | |

1-4 Hair rinse,
5-6 hair treatment

TABLE 11
Formulations for conditioners II.
Cosmetic preparations conditioners
(water, preservative to 100% by weight)

| Composition (INCI) | 7 | 8 | 9 | 10 |
|---|---|---|---|---|
| Texapon ® NSO | 38.0 | 38.0 | 25.0 | — |
| Sodium Laureth Sulfate | | | | |
| Texapon ® SB 3 | — | — | 10.0 | — |
| Disodium Laureth Sulfosuccinate | | | | |
| Plantacare ® 818 | 7.0 | 7.0 | 6.0 | — |
| Coco Glucosides | | | | |
| Plantacare ® PS 10 | — | — | — | 20.0 |
| Sodium Laureth Sulfate (and) Coco | | | | |
| Glucosides | | | | |
| Dehyton ® PK 45 | — | — | 10.0 | — |
| Cocamidopropyl Betaine | | | | |
| Lamesoft ® PO 65 | 3.0 | | | 4.0 |
| Coco-Glucoside (and) Glyceryl Oleate | | | | |
| Lamesoft ® LMG | — | 5.0 | — | — |
| Glyceryl Laurate (and) Potassium Cocoyl | | | | |
| Hydrolyzed Collagen | | | | |
| Euperlan ® PK 3000 AM | — | 3.0 | 5.0 | 5.0 |
| Glycol Distearate (and) Laureth-4 | | | | |
| (and) Cocamidopropyl Betaine | | | | |
| Mourera fluviatilis extract | 1.0 | 1.0 | 1.0 | 1.0 |
| Arlypon ® F | 3.0 | 3.0 | 1.0 | — |
| Laureth-2 | | | | |
| Sodium Chloride | — | 1.5 | — | 1.5 |

7-8 shower bath,
9 shower gel,
10 wash lotion

TABLE 12
Cosmetic preparations shampoo (water,
preservative to 100% by weight)

| Composition (INCI) | 15 | 16 | 17 | 18 | 19 | 20 |
|---|---|---|---|---|---|---|
| Texapon ® NSO | 30.0 | | | 30.0 | 25.0 | |
| Sodium Laureth Sulfate | | | | | | |
| Texapon ® K 14 S | | 30.0 | | | | 30.0 |
| Sodium Myreth Sulfate | | | | | | |
| Texapon ® SB 3 | | 10.0 | | | | |
| Disodium Laureth | | | | | | |
| Sulfosuccinate | | | | | | |
| Plantacare ® 818 | 4.0 | | | | | |
| Coca Glucosides | | | | | | |
| Plantacare ® 2000 | | 4.0 | | | | |
| Decyl Glucoside | | | | | | |
| Plantacare ® PS 10 | | | 20.0 | | | |
| Sodium Laureth | | | | | | |
| Sulfate (and) | | | | | | |
| Coca Glucosides | | | | | | |
| Dehyton ® PK 45 | 5.0 | | | 10.0 | | 10.0 |
| Cocamidopropyl Betaine | | | | | | |
| Gluadin ® WK | | | | | 8.0 | |
| Sodium Cocyl | | | | | | |
| Hydrolyzed Wheat Protein | | | | | | |
| Lamesoft ® PO 65 | — | — | — | — | 2.0 | 2.0 |
| Coco-Glucoside | | | | | | |
| (and) Glyceryl Oleate | | | | | | |
| Nutrilan ® Keratin W | 5.0 | — | — | — | | — |
| Hydrolyzed Keratin | | | | | | |
| Gluadin ® W 40 | — | 2.0 | — | 2.0 | — | — |
| Hydrolyzed Wheat Protein | | | | | | |
| Euperlan ® PK 3000 AM | — | — | — | 3.0 | 3.0 | — |
| Glycol Distearate | | | | | | |
| (and) Laureth-4 (and) | | | | | | |
| Cocamidopropyl | | | | | | |
| Betaine | | | | | | |
| Panthenol | — | — | — | — | — | 0.2 |
| Mourera fluviatilis extracts | 1.0 | 1.0 | 1.0 | 1.0 | 1.0 | 1.0 |
| Arlypon ® F | 1.5 | — | — | — | — | — |
| Laureth-2 | | | | | | |
| Sodium Chloride | — | 1.6 | 2.0 | 2.2 | — | 3.0 |

TABLE 13
Cosmetic preparations “2-in-1” shower bath
(water, preservative to 100%
by weight)

| Composition (INCI) | 11 | 12 | 13 | 14 |
|---|---|---|---|---|
| Texapon ® NSO | 30.0 | 25.0 | | 25.0 |
| Sodium Laureth Sulfate | | | | |
| Plantacare ® 818 | | | | 8.0 |
| Coco Glucosides | | | | |
| Plantacare ® 2000 | | 8.0 | | |
| Decyl Glucoside | | | | |
| Plantacare ® PS 10 | | | 20.0 | |
| Sodium Laureth Sulfate (and) Coco | | | | |
| Glucosides | | | | |
| Dehyton ® PK 45 | | 10.0 | 10.0 | |
| Cocamidopropyl Betaine | | | | |
| Lamesoft ® PO 65 | 5.0 | | | |
| Coco-Glucoside (and) Glyceryl Oleate | | | | |
| Lamesoft ® LMG | | 5.0 | 5.0 | |
| Glyceryl Laurate (and) Potassium Cocoyl | | | | |
| Hydrolyzed Collagen | | | | |
| Gluadin ® WQ | 3.0 | | | |
| Laurdimonium Hydroxypropyl Hydrolyzed | | | | |
| Wheat Protein | | | | |
| Gluadin ® WK | | | | |
| Sodium Cocoyl Hydrolyzed Wheat Protein | | | | |
| Euperlan ® PK 3000 AM | 5.0 | 3.0 | 4.0 | — |
| Glycol Distearate (and) Laureth-4 (and) | | | | |
| Cocamidopropyl Betaine | | | | |
| Panthenol | 0.5 | — | — | 0.5 |
| Mourera fluviatilis extracts | 1.0 | 1.0 | 1.0 | 1.0 |
| Arlypon ® F | 2.6 | 1.6 | — | 1.0 |
| Laureth-2 | | | | |
| Sodium Chloride | — | — | — | — |

TABLE 14
Cosmetic preparations foam bath
(water, preservative to 100% by weight)

| Composition (INCI) | 21 | 22 | 23 | 24 | 25 |
|---|---|---|---|---|---|
| Texapon ® NSO | — | 30.0 | 30.0 | — | 25.0 |
| Sodium Laureth Sulfate | | | | | |
| Plantacare ® 818 | — | 10.0 | — | — | 20.0 |
| Coca Glucosides | | | | | |
| Plantacare ® PS 10 | 22.0 | — | 5.0 | 22.0 | — |
| Sodium Laureth Sulfate | | | | | |
| (and) Coco Glucosides | | | | | |
| Dehyton ® PK 45 | 15.0 | 10.0 | 15.0 | 15.0 | 15.0 |
| Cocamidopropyl Betaine | | | | | |
| Monomuls ® 90-O 18 | 0.5 | | | | |
| Glyceryl Oleate | | | | | |
| Lamesoft ® PO 65 | | 3.0 | | 3.0 | 2.0 |
| Coco-Glucoside (and) Glyceryl | | | | | |
| Oleate | | | | | |
| Cetiol ® HE | | | 2.0 | | 2.0 |
| PEG-7 Glyceryl Cocoate | | | | | |
| Nutrilan ® I-50 | 5.0 | | | | |
| Hydrolyzed Collagen | | | | | |
| Gluadin ® W 40 | | 5.0 | | 5.0 | |
| Hydrolyzed Wheat Gluten | | | | | |
| Gluadin ® WK | | | | 7.0 | |
| Sodium Cocoyl Hydrolyzed Wheat | | | | | |
| Protein | | | | | |
| Euperlan ® PK 3000 AM | 5.0 | — | — | 5.0 | — |
| Glycol Distearate (and) Laureth-4 | | | | | |
| (and) Cocamidopropyl | | | | | |
| Betaine | | | | | |
| Arlypon ® F | | | 1.0 | | |
| Laureth-2 | | | | | |
| Sodium Chloride | 1.0 | | 1.0 | | 2.0 |
| Mourera fluviatilis extracts | 1.0 | 1.0 | 1.0 | 1.0 | 1.0 |

TABLE 15
Cosmetic preparations (water,
preservative to 100% by weight)

| Composition (INCI) | 31 | 32 | 33 | 34 | 35 | 36 | 37 | 38 | 39 | 40 |
|---|---|---|---|---|---|---|---|---|---|---|
| Dehymuls ® PGPH | 4.0 | 3.0 | — | 5.0 | — | — | — | — | — | — |
| Polyglyceryl-2 Dipolyhydroxystearate | | | | | | | | | | |
| Lameform ® TGI | 2.0 | 1.0 | — | — | — | — | — | — | — | — |
| Polyglyceryl-3 Diisostearate | | | | | | | | | | |
| Emulgade ® PL 68/50 | — | — | — | — | 4.0 | — | — | — | 3.0 | — |
| Cetearyl Glucoside | | | | | | | | | | |
| (and) Cetearyl Alcohol | | | | | | | | | | |
| Eumulgin ® B2 | — | — | — | — | — | — | — | 2.0 | — | — |
| Ceteareth-20 | | | | | | | | | | |
| Tegocare ® PS | — | — | 3.0 | — | — | — | 4.0 | — | — | — |
| Polyglyceryl-3 | | | | | | | | | | |
| Methylglucose Distearate | | | | | | | | | | |
| Eumulgin VL 75 | — | — | — | — | — | 3.5 | — | — | 2.5 | — |
| Polyglyceryl-2 Dipolyhydroxystearate | | | | | | | | | | |
| (and) Lauryl Glucoside (and) Glycerin | | | | | | | | | | |
| Bees Wax | 3.0 | 2.0 | 5.0 | 2.0 | — | — | — | — | — | — |
| Cutina ® GMS | — | — | — | — | — | 2.0 | 4.0 | — | — | 4.0 |
| Glyceryl Stearate | | | | | | | | | | |
| Lanette ® O | — | — | 2.0 | — | 2.0 | 4.0 | 2.0 | 4.0 | 4.0 | 1.0 |
| Cetearyl Alcohol | | | | | | | | | | |
| Antaron ® V 216 | — | — | — | — | — | 3.0 | — | — | — | 2.0 |
| PVP/Hexadecene Copolymer | | | | | | | | | | |
| Myritol ® 818 | 5.0 | — | 10.0 | — | 8.0 | 6.0 | 6.0 | — | 5.0 | 5.0 |
| Cocoglycerides | | | | | | | | | | |
| Finsolv ® TN | — | 6.0 | — | 2.0 | — | — | 3.0 | — | — | 2.0 |
| C12/15 Alkyl Benzoate | | | | | | | | | | |
| Cetiol ® J 600 | 7.0 | 4.0 | 3.0 | 5.0 | 4.0 | 3.0 | 3.0 | — | 5.0 | 4.0 |
| Oleyl Erucate | | | | | | | | | | |
| Cetiol ® OE | 3.0 | — | 6.0 | 8.0 | 6.0 | 5.0 | 4.0 | 3.0 | 4.0 | 6.0 |
| Dicaprylyl Ether | | | | | | | | | | |
| Mineral Oil | — | 4.0 | — | 4.0 | — | 2.0 | — | 1.0 | — | — |
| Cetiol ® PGL | — | 7.0 | 3.0 | 7.0 | 4.0 | — | — | — | 1.0 | — |
| Hexadecanol (and) Hexyldecyl Laurate | | | | | | | | | | |
| Panthenol/Bisabolol | 1.2 | 1.2 | 1.2 | 1.2 | 1.2 | 1.2 | 1.2 | 1.2 | 1.2 | 1.2 |
| Mourera fluviatilis extract (Example 3,4 | 1.0 | 1.0 | 1.0 | 1.0 | 1.0 | 1.0 | 1.0 | 1.0 | 1.0 | 1.0 |
| or 5) | | | | | | | | | | |
| Copherol ® F 1300 | 0.5 | 1.0 | 1.0 | 2.0 | 1.0 | 1.0 | 1.0 | 2.0 | 0.5 | 2.0 |
| Tocopherol/Tocopheryl Acetate | | | | | | | | | | |
| Neo Heliopan ® Hydro | 3.0 | — | — | 3.0 | — | — | 2.0 | — | 2.0 | — |
| Sodium Phenylbenzimidazole Sulfonate | | | | | | | | | | |
| Neo Heliopan ® 303 | — | 5.0 | — | — | — | 4.0 | 5.0 | — | — | 10.0 |
| Octocrylene | | | | | | | | | | |
| Neo Heliopan ® BB | 1.5 | — | — | 2.0 | 1.5 | — | — | — | 2.0 | — |
| Benzophenone-3 | | | | | | | | | | |
| Neo Heliopan ® E 1000 | 5.0 | — | 4.0 | — | 2.0 | 2.0 | 4.0 | 10.0 | — | — |
| Isoamyl p-Methoxycinnamate | | | | | | | | | | |
| Neo Heliopan ® AV | 4.0 | — | 4.0 | 3.0 | 2.0 | 3.0 | 4.0 | — | 10.0 | 2.0 |
| Octyl Methoxycinnamate | | | | | | | | | | |
| Uvinul ® T 150 | 2.0 | 4.0 | 3.0 | 1.0 | 1.0 | 1.0 | 4.0 | 3.0 | 3.0 | 3.0 |
| Octyl Triazone | | | | | | | | | | |
| Zinc Oxide | — | 6.0 | 6.0 | — | 4.0 | — | — | — | — | 5.0 |
| Titanium Dioxide | — | — | — | — | — | — | — | 5.0 | — | — |
| Glycerol (86% by weight) | 5.0 | 5.0 | 5.0 | 5.0 | 5.0 | 5.0 | 5.0 | 5.0 | 5.0 | 5.0 |

31 w/o sun protection cream,
32-34 w/o sun protection lotion,
35,38,40 o/w sun protection lotion,
36,37,39 o/w sun protection cream

1. Extracts of the plant Mourera fluviatilis.

2. Extract as claimed in claim 1, characterized in that solvents or mixtures of solvents selected from the group consisting of distilled or nondistilled water, low molecular weight alcohols, esters, hydrocarbons, ketones or halogen-containing hydrocarbons are used as the extraction medium.

3. Cosmetic and/or pharmaceutical preparations, characterized in that they contain an extract of the plant Mourera fluviatilis.

4. Preparations as claimed in claim 3, characterized in that they contain the plant extract in quantities of 0.01 to 25% by weight, based on the final preparation, with the proviso that the quantities mentioned add up to 100% by weight with water and optionally other auxiliaries and additives.

5. Preparations as claimed in at least one of claims 3 and/or 4, characterized in that the extract contains substances selected from the group consisting of saponins, flavone derivatives, sterols, triterpenes, xanthone derivatives and carotinoids.

6. Preparations as claimed in at least one of claims 3 and/or 4, characterized in that the extract contains salts selected from the group consisting of salts of the alkali and alkaline earth metals, salts of sodium, potassium and/or calcium being particularly preferred.

7. The use of extracts of the plant Mourera fluviatilis in skin and/or hair care preparations.

8. The use of extracts of the plant Mourera fluviatilis in moisture-regulating moisturizers.

9. The use of extracts of the plant Mourera fluviatilis in sun protection preparations.

10. The use of extracts of the plant Mourera fluviatilis in preparations against fibroblast and/or keratinocyte damage by UV-A radiation and/or UV-B radiation.

11. The use of extracts of the plant Mourera fluviatilis as anti-inflammatory agents.

12. The use of extracts of the plant Mourera fluviatilis as antioxidants.

13. A process for the preparation of an extract of the plant Mourera fluviatilis, characterized in that solvents or mixtures of solvents selected from the group consisting of distilled or nondistilled water, low molecular weight alcohols, esters, hydrocarbons, ketones or halogen-containing hydrocarbons are used for extraction.
